(19) United States
(12) Patent Application Publication
(10) Pub. No.: US 2006/0025801 A1
(43) Pub. Date: Feb. 2, 2006

(54) Embolic device deployment system with filament release

(76) Inventors: Robert Lulo, Pembroke Pines, FL (US); Darren Sherman, Ft. Lauderdale, FL (US); Damian Tomlin, Pembroke Pines, FL (US)

Correspondence Address: Philip S. Johnson, Esq.;Johnson & Johnson, One Johnson & Johnson Plaza, New Brunswick, NJ, 08933-7003, US

(21) Appl. No.: 11/145,350

(22) Filed: Jun. 3, 2005

Related U.S. Application Data
(60) Provisional application No. 60/592,580, filed on Jul. 30, 2004.

(51) Int. Cl. A61M 29/00

(52) U.S. Cl. 606/200

(57) ABSTRACT

A medical device for placing an embolic device, such as an embolic coil, at a predetermined site within a vessel of the body including a delivery catheter and a flexible pusher member movably disposed within the catheter. An embolic device is detachably coupled to the pusher member and is retained in place on the distal end of the pusher member by a detachment filament. When the embolic device is advanced to the predetermined site within the vessel, the detachment filament is decoupled from the embolic device to thereby release the embolic device.

### CROSS-REFERENCE TO RELATED APPLICATION(S)

This patent application claims priority from Provisional Patent Application Ser. No. 60/592,580, filed on Jul. 30, 2004.

### BACKGROUND OF INVENTION

1. Field of the Invention

The present invention relates to a medical device for placing an embolic device at a predetermined site within a vessel of the human body, and more particularly, relates to a catheter-based deployment system for delivering an embolic device. This device is particularly suited to transport an embolic device, such as an embolic coil, through the tortious vasculature of the human brain to a selected site.

2. Description of the Prior Art

For many years, flexible catheters have been used to place various devices within the vessels of the human body. Such devices include dilation balloons, radiopaque fluids, liquid medications, and various types of occlusion devices such as balloons and embolic coils. Examples of such catheter-based devices are disclosed in U.S. Pat. No. 5,108,407, entitled, “Method And Apparatus For Placement Of An Embolic Coil,” and U.S. Pat. No. 5,122,136, entitled, “Endovascular Electrolytically Detachable Guidewire Tip For The Electroformation Of Thrombus In Arteries, Veins, Aneurysms, Vascular Malformations And Arteriovenous Fistulas.”0 These patents disclose catheter-based devices for delivering embolic coils to preselected positions within vessels of the human body in order to treat aneurysms, or alternatively, to occlude blood vessels at a particular location.

Coils which are placed in vessels may take the form of helically wound coils, or alternatively, may take the form of randomly wound coils, coils wound within coils or other such coil configurations. Examples of various coil configurations are disclosed in U.S. Pat. No. 5,334,210, entitled “Vascular Occlusion Assembly;” and U.S. Pat. No. 5,382,259, entitled, “Vasoocclusion Coil with Attached Tubular Woven or Braided Fibrous Covering.” Embolic coils are generally formed of a radiopaque metallic material, such as platinum, gold, tungsten, or alloys of these metals. Often, several coils are placed at a given location to occlude the flow of blood through the vessel, or aneurysm, by promoting thrombus formation at the particular site.

In the past, embolic coils have been placed within the distal end of a catheter. When the distal end of the catheter is properly positioned, the coil may then be pushed out of the end of the catheter with a pusher member to release the coil at the desired location. This procedure for placement of an embolic coil is conducted under fluoroscopic visualization such that the movement of the coil through the vasculature of the body may be monitored and the coil placed at the desired location.

Another procedure involves the use of glue or solder for attaching the coil to a guidewire, which in turn, is placed within a flexible catheter for positioning the coil within the vessel at a preselected position. Once the coil is in the desired position, the coil is held in position by the catheter and the guidewire is pulled proximally to thereby cause the coil to become detached from the guidewire and released from the catheter. Such a coil positioning system is disclosed in U.S. Pat. No. 5,263,964 entitled, “Coaxial Traction Detachment Apparatus and Method.”

Still another coil positioning procedure is that of having a catheter with a socket at the distal end of the catheter for retaining a ball which is, in turn, bonded to the proximal end of the coil. The ball, which is generally larger in diameter than the outside diameter of the coil, is placed in the socket within the lumen at the distal end of the catheter and the catheter is then moved into a vessel in order to place the coil at a desired position. Once the position is reached, a pusher wire with a piston at the end thereof is pushed distally from the proximal end of the catheter to push the ball out of the socket in order to release the coil at the desired position. Such a system is disclosed in U.S. Pat. No. 5,350,397, entitled, “Axially Detachable Embolic Coil Assembly.”

Another procedure for placing an embolic coil within a vessel is that of using a heat releasable adhesive bond for retaining the coil at the distal end of the catheter. One such system uses laser energy transmitted through a fiber optic cable to apply heat to the adhesive bond in order to release the coil from the end of the catheter. Such a procedure is disclosed in the aforementioned U.S. Pat. No. 5,108,407.

Yet another coil deployment system incorporates a catheter having a lumen throughout the length of the catheter and a distal tip for retaining the coil for positioning the coil at a preselected site. The distal tip of the catheter is formed of a material which exhibits the characteristic that when the lumen of the catheter is pressurized the distal tip expands radially to release the coil at the preselected site. Such a deployment system is disclosed in U.S. Pat. No. 6,113,622, entitled, “Embolic Coil Hydraulic Deployment System.”

Still another coil deployment system incorporates an interlocking mechanism on the coil. The interlocking end on the embolic coil couples with a similar interlocking mechanism on a pusher assembly. A control wire which extends through the locking mechanism secures the coil to the pusher assembly. The pusher assembly and embolic coil are initially disposed within the lumen of a catheter. When the embolic coil is pushed out of the end of the catheter for placement, the control wire is retracted and the coil disengages from the pusher assembly. Such a deployment system is disclosed in U.S. Pat. No. 5,925,059, entitled, “Detachable Embolic Coil Assembly.”

Yet another coil deployment system incorporates an embolic device detachably mounted on the distal portion of a pusher member and held in place with a connector thread or fiber. The fiber passes through a cutter member that may be activated to cut the connector fiber. Once the connector fiber is cut, the embolic device is released. Such a deployment system is disclosed in Published U.S. patent application No. 2002/0165569, entitled, “Intravascular Device Deployment Mechanism Incorporating Mechanical Detachment.”

Still another coil deployment system incorporates an embolic device with a stretch resistant member therethrough. The distal end of the stretch resistant member attaches to the embolic coil and the proximal end of the stretch resistant member is detachably mounted on the pusher member through various means such as adhesive, or by a connector fiber adhered to or tied to the pusher member, and is detachable by the application of heat. Such a deployment system is disclosed in Published U.S. patent application No. 2004/0034363, entitled, “Stretch Resistant Therapeutic Device.”

Still another coil deployment system incorporates a pusher wire with a stiff wavy-shaped end segment which is coupled to the embolic coil and is placed in the lumen of the catheter. The coil is advanced through the catheter until it reaches a predetermined site in the vessel at which time the pusher wire is retracted and the embolic coil is released. Such a system is disclosed in U.S. Pat. No. 6,203,547, entitled, “Vaso-occlusion Apparatus Having A Manipulable Mechanical Detachment Joint And A Method For Using The Apparatus.”

Still another embolic device deployment system includes an elongated flexible pusher member slidably disposed within a lumen of a catheter. The embolic device is retained at the end of the pusher member with a detachment filament. When the embolic device is advanced to the predetermined site within the vessel, the detachment filament is withdrawn releasing the embolic device.

### SUMMARY OF THE INVENTION

The present invention is directed toward a vasooclusive embolic device deployment system for use in placing an embolic device at a predetermined site within a vessel including an elongated flexible catheter and an elongated pusher member, preferably having a lumen therethrough and being slidably disposed within the catheter. An embolic device, preferably taking the form of a helically wound embolic coil having a plurality of turns, is releasably coupled to the distal end of the pusher member. The detachment system also includes a detachment filament which extends through the lumen of the catheter, or preferably through the lumen of the pusher member. The detachment filament extends around a turn of the embolic coil and then back through the lumen of the catheter, or preferably through a lumen of the pusher member. When the embolic coil is at the predetermined site within the vessel, the detachment filament may be pulled proximally to decouple the detachment filament from engagement of the turn of the embolic coil to thereby release the embolic coil.

In accordance with another aspect of the present invention, the vasooclusive embolic device deployment system includes a retaining clamp assembly mounted on the proximal end of the pusher member. The retaining clamp preferably takes the form of an adjustable chuck which applies a clamping pressure to the fiber to retain the filament until the chuck is opened to release the filament. The detachment filament extends through the clamp then through and the lumen of the pusher member and then around a turn of the embolic coil, and then back through the lumen of the pusher member and through the retaining clamp. Upon loosening the retaining clamp, one of the ends of the detachment filament may be pulled proximally to decouple the detachment filament from the embolic coil to thereby release the embolic coil at the predetermined site.

These and other aspects of the present invention and the advantages thereof will be more clearly understood from the following description and drawings of a preferred embodiment of the present invention:

### BRIEF DESCRIPTION OF THE DRAWINGS

FIG. 1 is an enlarged, partially sectional view of an embodiment of an embolic device deployment system in accordance with the present invention;

FIG. 2 is an enlarged, sectional view, illustrating in more detail the coil deployment system of FIG. 1;

FIGS. 3, 3a, 3b, and 3c are enlarged, sectional views of the coil deployment system shown in FIGS. 1 and 2 illustrating the sequential steps in the advancement of the embolic device, removal of a detachment filament, and release of the embolic device.

### DESCRIPTION OF THE PREFERRED EMBODIMENT

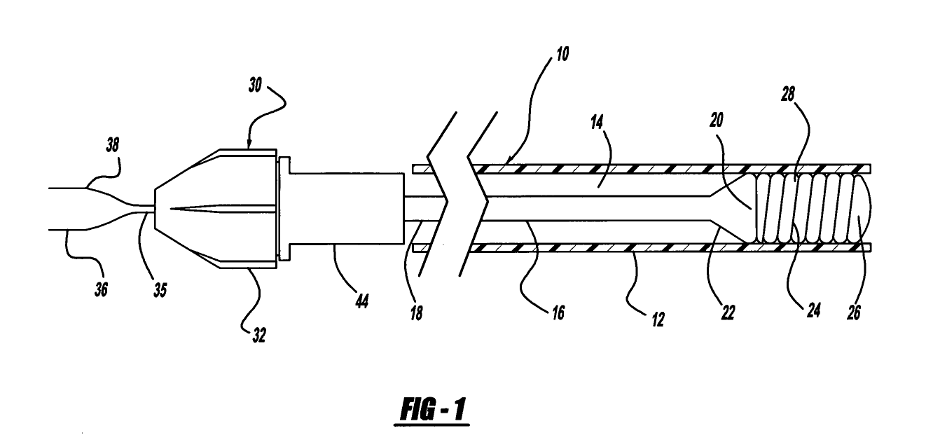

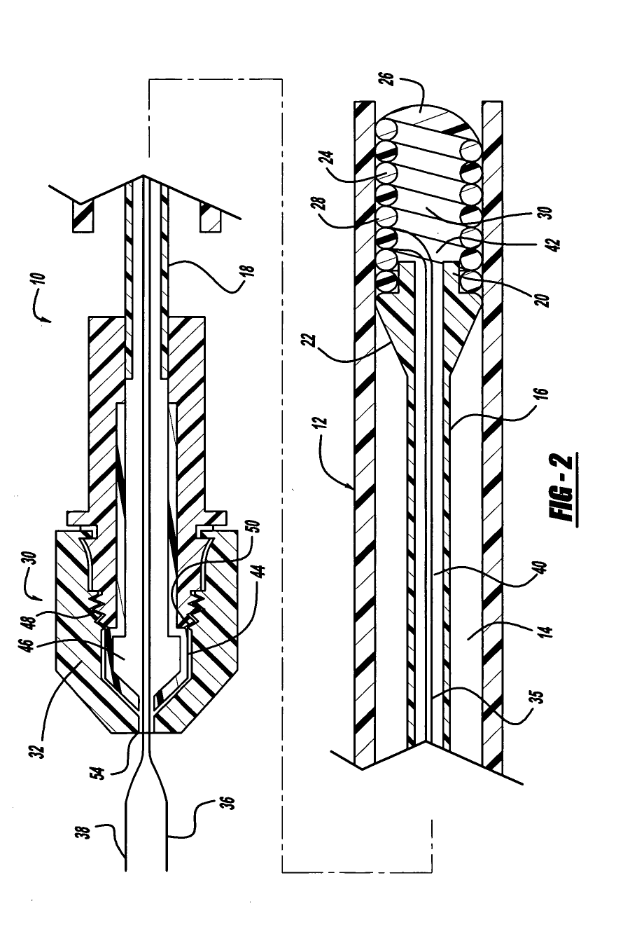

FIG. 1 generally illustrates one embodiment of an embolic device deployment system 10 of the present invention having an elongated flexible catheter 12 having a lumen 14 extending therethrough. An elongated flexible pusher member 16 is slidably disposed within the lumen 14 of the catheter 12. The pusher member 16 includes a proximal end 18 and a distal end 20, and the distal end 20 includes a tip portion 22 having an increase in diameter. Retractably mounted on the pusher member 16 at its distal end 20 is an embolic device, which preferably takes the form of an embolic coil 24, formed of a plurality of helical turns 28 connected to an atraumatic distal bead 26. While the embolic coil 24 is a preferred configuration of the embolic device, alternative device configurations are suitable such as embolic filaments, braids, expandable meshes, foams and stents. The tip portion 22 at the distal end 20 of the pusher member 16 engages the embolic coil 24. Mounted on the proximal end 18 of the pusher member 16 is a retaining clamp assembly 30, which includes a cap 32 which engages a chuck 44. A detachment filament 35 having ends 36 and 38 extends through the retaining clamp assembly 30 and serves to retain the embolic coil 24 in position at the distal end 20 of the elongated pusher member 16.

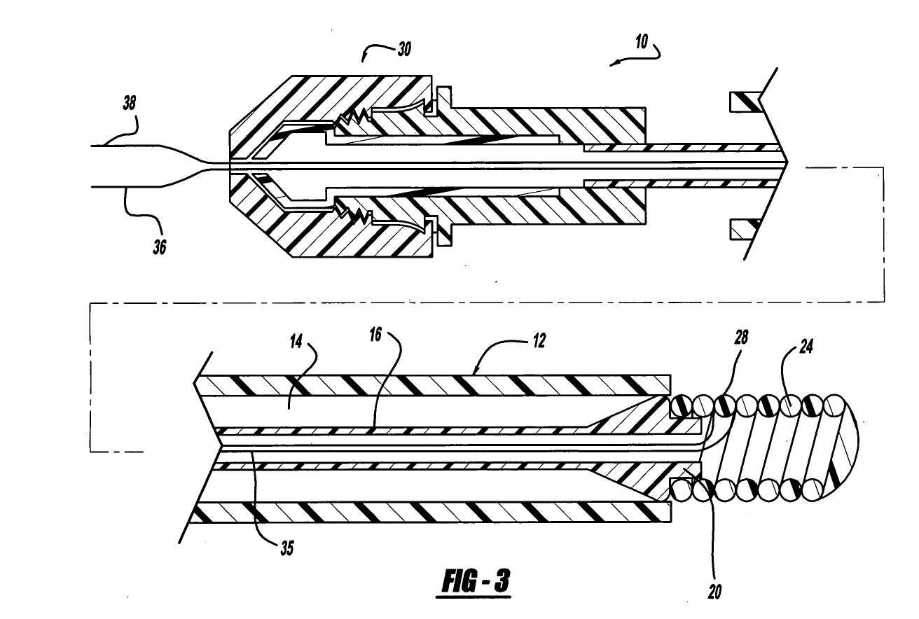

FIG. 2 illustrates in more detail the configuration of the embolic device deployment system 10 of FIG. 1. The pusher member 16, preferably has a lumen 40 therethrough and is slidably disposed within the lumen 14 of the catheter 12. Preferably, the pusher member 16 is constructed from nitinol, but alternatively, the pusher member may be constructed from many materials that are pushable and flexible such as stainless steel, nylon, PTFE, other metals or polymers and composites. Additionally, the pusher member 16 should have an outside diameter in the range of about 0.002 to 0.020 centimeters.

As shown, the embolic coil 24 is helically wound but may take various other forms. Such as for example, a randomly wound coil. The distal bead 26 is connected to the distal end of the embolic coil 24. The embolic coil 24 also includes a lumen 42 extending therethrough created by the plurality of helical turns 28. The diameter of the lumen 42 of the embolic coil 24 is slightly greater than the diameter of the distal end 20 of the pusher member 16. With this configuration, the embolic coil 24 may be pushed distally by the distal end 20 of the pusher member 16.

A detachment filament 35 includes ends 36 and 38 which extend proximally from the proximal end of the clamp assembly 30. The detachment filament 35 extends through the retaining clamp assembly 30 and through the lumen 40 of the pusher member 16. The detachment filament 35 also loops around one of the plurality of helical turns 28 of the embolic coil 24 and is returned through the lumen 40 of the pusher member 16, exits the pusher member 16 and extends through the lumen 46 of the chuck 44 of the clamp assembly 30 and then exits at the proximal end of the clamp assembly 30. The cap 32 applies pressure to the chuck 44, such that the chuck 44 applies squeezing pressure to the ends 36 and 38 of the detachment filament 35 thereby preventing movement of the detachment filament 35. The embolic coil 24 may not be disengaged from the distal end 20 of the pusher member 16, so long as the cap 32 remains tight on the chuck 44 and the detachment filament 35 is secured relative to the pusher member 16. Finally, the detachment filament 35 is preferably constructed of nitinol, but alternatively may be formed from various other materials such as platinum, nylon, PTFE, flexible metals, polymers, or composites. Preferably, the material used for the detachment filament 35should be very flexible, have a high tensile strength and a low elongation when a tensile force is applied to the filament. The diameter of the detachment filament 35 is in the range of about 0.001 to 0.090 centimeters and preferably on the order of about 0.002 to 0.020 centimeters.

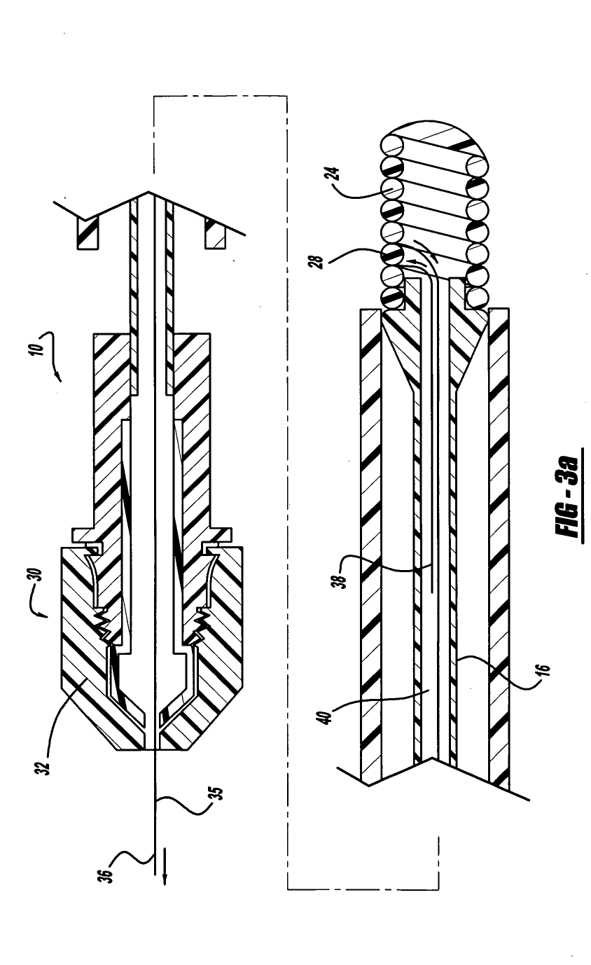

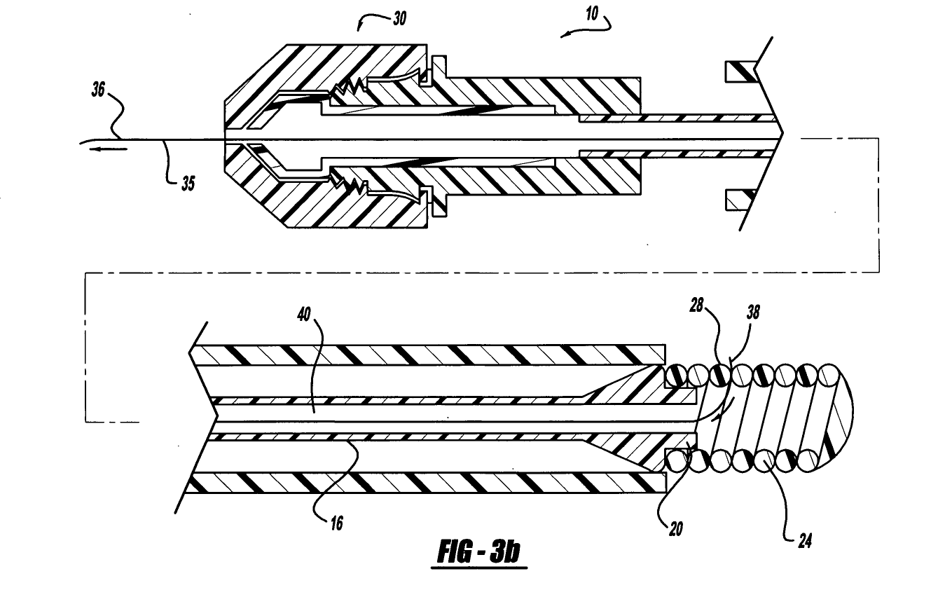

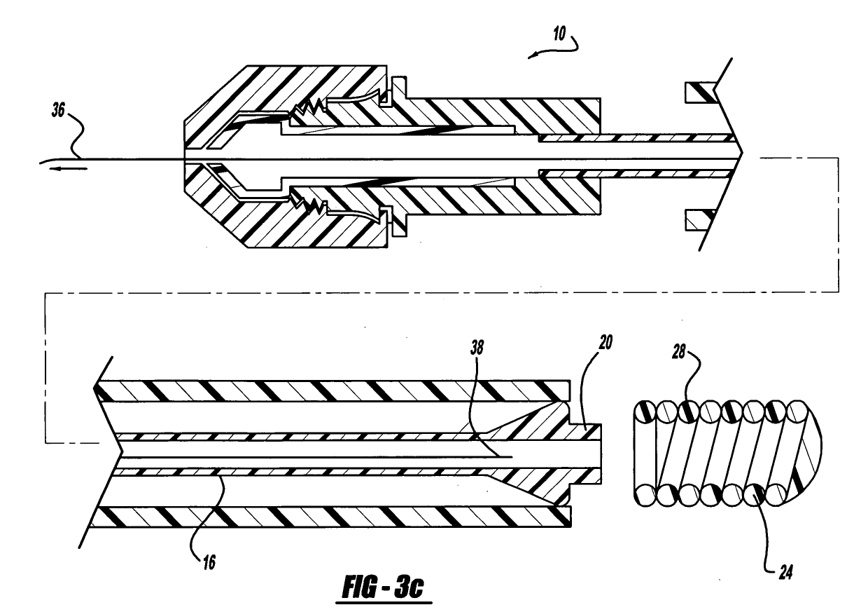

FIGS. 3, 3a, 3b, and 3c generally illustrate the operation of the embolic device deployment system 10 and demonstrate the detachment filament release mechanism. More particularly, FIG. 3 illustrates the catheter 12 positioned at a predetermined location, analogous to placement in a vessel and the pusher member 16 advanced through the lumen 14 of the catheter 12, such that the embolic coil 24 exits the distal end of the catheter 12. In addition, the retaining clamp assembly 30 maintains tension on the ends 36 and 38 of detachment filament 35, such that the embolic coil 24 is retained at the distal end 20 of the pusher member 16.

FIG. 3a illustrates the embolic device deployment system 10 with the embolic coil 24 positioned at a desired location adjacent the distal section of the catheter 12. The cap 32 is loosened to permit one end 36 of the detachment filament 35 to be pulled proximally. As the end 36 of the detachment filament 35 is pulled proximally from the retaining clamp assembly 30, the other end 38 of the detachment filament 35 moves distally through the lumen 40 of the pusher member 16.

FIG. 3b illustrates the embolic device deployment system 10 with the end 36 of the detachment filament 35 pulled further proximally from the retaining clamp assembly 30 and the other end 38 of the detachment filament 35 withdrawn from its position around one of the plurality of helical turns 28 of the embolic coil 24.

FIG. 3c illustrates the embolic device deployment system 10 with the end 38 of the detachment filament 35 completely removed from the helical turn 28 of the embolic coil 24. Finally, the embolic coil 24 disengages from the distal end 20 of the pusher member 16 and is released at the predetermined site within the vessel.

As is apparent, there are numerous modifications of the preferred embodiment described above which will be readily apparent to one skilled in the art, such as many variations and modifications of the coil including numerous coil winding configurations, or alternatively other types of implant devices. There are obviously variations in the path and attachment of the detachment filament. Additionally, the retaining clamp assembly could also be modified with other methods used to apply pressure to the detachment filament ends. These modifications would be apparent to those having ordinary skill in the art to which this invention relates and are intended to be within the scope of the claims which follow.

That which is claimed is:

1. A vasooclusive embolic device deployment system for use in placing an embolic device at a predetermined site within a vessel comprising:
an elongated flexible catheter having a lumen extending therethrough and having proximal and distal ends;
an elongated pusher member having a lumen therethrough having proximal and distal ends and being slidably disposed within the lumen of the catheter;
an embolic device having a plurality of turns releasably engaging the distal end of the pusher member; and,
a detachment filament extending from a position proximal of the proximal end of the catheter through the lumen of the pusher member around a turn of the embolic device and extending back through the lumen of the pusher member to a position proximal of the catheter, such that when the embolic device is properly positioned at a predetermined site within the vessel the detachment filament may be pulled proximally to decouple the detachment filament from the turn of the embolic device to thereby release the embolic device at the predetermined site.

2. A vasooclusive embolic device deployment system as defined in claim 1, including a retaining clamp having a lumen extending therethrough and being mounted on the proximal end of the pusher member, and wherein said detachment filament extends from a position proximal of the proximal end of the clamp through the lumen of the clamp, through the lumen of the pusher member around the turn of the embolic device and then back through the lumen of the pusher member and through the lumen of the clamp to a position proximal of the proximal end of the clamp so that upon release of the clamp the detachment filament may be withdrawn proximally to release the embolic device.

3. A vasooclusive embolic device deployment system as defined in claim 2, including the retaining clamp comprising a chuck having a tightening cap for controlling pressure applied to the detachment filament.

4. A vasooclusive embolic device deployment system as defined in claim 1, wherein the detachment filament is formed of nitinol.

5. A vasooclusive embolic device deployment system as defined in claim 1, wherein the embolic device is helically wound.

6. A vasooclusive embolic device deployment system for use in placing an embolic device at a predetermined site within a vessel comprising:
an elongated flexible catheter having a lumen extending therethrough and having proximal and distal ends;
an elongated pusher member having a lumen therethrough having proximal and distal ends and being slidably disposed within the lumen of the catheter;
an embolic device having a plurality of turns releasably engaging the distal end of the pusher member;
a retaining clamp having a lumen extending therethrough and being mounted on the proximal end of the pusher member; and,
a detachment filament extending from a position proximal of the clamp through the lumen of said clamp and through the lumen of the pusher member and looping around a turn of the embolic device such that when the embolic device is properly positioned at the predetermined site within the vessel the detachment filament may be pulled proximally to decouple the detachment filament from the turn of the embolic device to thereby release the embolic device at the predetermined site.

7. A vasooclusive embolic device deployment system as defined in claim 6, wherein the retaining clamp comprises an adjustable chuck.

8. A vasooclusive embolic device deployment system as defined in claim 6, wherein the detachment filament is formed of nitinol.

9. A vasooclusive embolic device deployment system as defined in claim 6, wherein the embolic device is a helically wound embolic coil.

10. A vasooclusive embolic device deployment system for use in placing an embolic device at a predetermined site within a vessel comprising:
an elongated flexible catheter having a lumen extending therethrough and having proximal and distal ends;
an elongated pusher member having a lumen extending therethrough and having proximal and distal ends, said elongated pusher member being slidably disposed within the lumen of the catheter;
an embolic device releasably engaging the distal end of the pusher member;
a retaining clamp having a lumen extending therethrough and being mounted to the proximal end of the pusher member; and,
a detachment filament extending from a position proximal of the clamp through the lumen of the clamp and through the lumen of the pusher member and extending through a passageway in the embolic device, such that when the embolic device is properly positioned at the predetermined site within the vessel the detachment filament may be pulled proximally to decouple the detachment filament from the embolic device to thereby release the embolic device into the vessel.

11. A vasooclusive embolic device deployment system as defined in claim 10, wherein the retaining clamp comprises an adjustable chuck.

12. A vasooclusive embolic device deployment system as defined in claim 10, wherein the detachment filament is formed of nitinol.

13. A vasooclusive embolic device deployment system as defined in claim 10, wherein the embolic device is an embolic coil.

14. A vasooclusive embolic device deployment system as defined in claim 13, wherein the embolic coil is helically wound.

15. A vasooclusive embolic device deployment system for use in placing an embolic device at a predetermined site within a vessel comprising:
an elongated flexible catheter having a lumen extending therethrough and having proximal and distal ends;
an elongated pusher member having a lumen therethrough having proximal and distal ends and being slidably disposed within the lumen of the catheter;
an embolic device having a plurality of turns, said embolic device releasably engaging the distal end of the pusher member;
a retaining clamp having a lumen extending therethrough and being mounted on the proximal end of the pusher member; and,
a detachment filament extending from a position proximal of the clamp through the lumen of the clamp and through the lumen of the catheter and looping around a turn of the embolic device, such that when the embolic device is properly positioned at the predetermined site within the vessel the detachment filament may be pulled proximally to decouple the detachment filament from the turn of the embolic device to thereby release the embolic device at the predetermined site.

16. A vasooclusive embolic device deployment system as defined in claim 15, wherein the embolic device is an embolic coil.

17. A vasooclusive embolic device deployment system as defined in claim 16, wherein the embolic coil is helically wound.

18. A vasooclusive embolic device deployment system for use in placing an embolic device at a predetermined site within a vessel comprising:
an elongated flexible catheter having proximal and distal ends and a lumen extending therethrough and;
an elongated pusher member having proximal and distal ends and being slidably disposed within the lumen of the catheter;
an embolic device having proximal and distal ends, said embolic device releasably engaging the distal end of said elongated pusher member;
a retaining clamp being positioned adjacent to and engaging the proximal end of said pusher member; and,
a detachment filament extending from a position proximal of said retaining clamp, releasably secured by said retaining clamp and through the lumen of the catheter and looping around a portion of the proximal end of said embolic device, such that when said embolic device is properly positioned at the predetermined site within the vessel, said detachment filament may be pulled proximally to decouple the detachment filament from the portion of the proximal end of the embolic device to thereby release said embolic device at the predetermined site.

19. A vasooclusive embolic device deployment system as defined in claim 18, wherein the embolic device is an embolic coil.

20. A vasooclusive embolic device deployment system as defined in claim 19, wherein the embolic coil is helically wound.
